(19) United States
(12) Patent Application Publication
(10) Pub. No.: US 2006/0155247 A1
(43) Pub. Date: Jul. 13, 2006

(54) Introducer sheath with rotatable stop cock

(76) Inventors: Bryan R. Lampropoulos, Sandy, UT (US); Fred P. Lampropoulos, Sandy, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 11/035,340

(22) Filed: Jan. 13, 2005

(51) Int. Cl. A61M 5/178

(52) U.S. Cl. 604/167.05; 251/304

(57) ABSTRACT

An introducer sheath having a main body, a sheath, one or more connections to one or more corresponding entry lines, and a stop cock delivery valve. The stop cock can connect to at least one of the entry lines, and can further connect to a fluid source, thereby providing a manipuable delivery interface from the fluid source to the introducer sheath housing. A rotator can interface at a point between the introducer sheath and the stop cock, allowing a user to configure the stop cock to conformations that can be easily accessible for right or left handed users, as appropriate for the position of the introducer sheath housing.

### CROSS-REFERENCE TO RELATED APPLICATIONS

This application claims the benefit of priority to U.S. patent application Ser. No. 11/021,513, filed on Dec. 23, 2004, entitled “Rotatable Suture Ring,” the entire specification of which application is incorporated herein by reference.

### BACKGROUND OF THE INVENTION

1. The Field of the Invention

This invention relates to a medical introducer sheath. In more particular, the present invention relates to methods and apparatuses for utilizing a rotator stop cock combination with an introducer sheath.

2. Background and Relevant Art

Present medical technologies include invasive and non-invasive devices for providing patients with localized medical treatment. One such invasive device is called an “introducer sheath,” which can be geared primarily for providing user (e.g., medical service provider) access to the inside of a patient's body, such as through a patient's artery or vein. Once a user gains such access, the introducer sheath allows the user to provide the patient with medical treatment such as by inserting additional tools through the introducer sheath, or through delivering certain fluids through the introducer sheath.

FIGS. 1A and 1B illustrate perspective views of a prior art introducer sheath 100 for use in combination with a stop cock 130. Introducer sheath 100 provides access to the vasculature or other internal portion of a patient's body. Introducer sheath 110 includes a main body 102, an introducer bore 105, a sheath 110, and a side port 112. A medical practitioner accesses the vasculature of the patient by inserting a guide wire, other tool, or implement into introducer bore 105, through the hemostasis valve (not shown) associated with introducer bore 105, into the lumen of main body 102, and down the length of sheath 110.

Stop cock 130 is connected to side port 112 of introducer sheath 100 utilizing entry line 120. The configuration of side port 112 permits the introduction of medicines, saline, or other fluids and materials into the patient through introducer sheath 100. Stop cock 130 allows a practitioner to control the flow of such fluids and materials being introduced through side port 112. Stop cock 130 includes a rotatable knob 135, an inlet port 132, an outlet port 134, and a side port 137. Rotatable knob 135 controls the flow of fluids and materials between inlet port 132 and an outlet port 134 of stop cock 130. For example, as fluid flows from entry line 125 to inlet port 132, a user can open or close fluid flow from inlet port 132 to outlet port 134. This also controls the flow of fluid to introducer sheath 100 by means of side port 112 and entry line 120.

As fluid flows through stop cock 130 into the introducer sheath 100, the user can move rotatable knob 135 to a stop position. When rotatable knob 135 is in a stop position, the flow of fluids to introducer sheath 100 is stopped. The user can stop the flow of fluids for a variety of reasons including preparation for removing and repositioning the introducer sheath in the vasculature of the patient, switching of infusate lines, or at the end of a procedure. When the practitioner is ready to resume the flow of fluids, the practitioner can turn rotatable knob 135 to a flow position. In the flow position, rotatable knob 135 allows fluids to flow from inlet port 132, to outlet port 134, to introducer sheath 100, and then into the patient.

One will appreciate that the position of the introducer sheath on a given patient can have a significant impact on the ease by which a user can access the rotatable knob 135, and hence, adjust the valve of stop cock 130. For example, in FIG. 1A stop cock 130 is configured for use when side port 112 is on the right hand side of introducer sheath 100. When side port 112 is positioned on the right hand side of introducer sheath 100, entry line 120 and stop cock 130 are also positioned on the right hand side of introducer sheath 100. In the illustrated embodiment, the configuration of stop cock 130 positions rotatable knob 135 facing to the front or upwards when side port 112, entry line 120, and stop cock 130 are positioned on the right side of introducer sheath 100. The front facing position of rotatable knob 135 is easily accessed and manipulated by the user during a medical procedure.

The illustrated positioning of rotatable knob 135, stop cock 130, and introducer sheath 100 can occur where introducer sheath 100 has been inserted into a patient's left arm, the patient lying face up, facing the practitioner. The positioning of introducer sheath 100 provides advantageous manipulation of rotatable knob 135 for a right handed user standing to the side of the patient. In the illustrated embodiment, stop cock 130 is a right handed stop cock that is configured specifically to be used in the illustrated, or other similar, setting.

FIG. 1B illustrates the positioning of rotatable knob 135, stop cock 130, and introducer sheath 100 when introducer sheath 100 is rotated 180 degrees from the positioning of FIG. 1A. In the illustrated configuration, side port 112 of introducer sheath 100 is positioned on the left side of introducer sheath 100. When side port 112 is positioned on the left side of introducer sheath 100, entry line 120 and stop cock 130 are also positioned on the left side of introducer sheath 100. However, as discussed with reference to FIG. 1A, stop cock 130 is a right handed stop cock. A rear surface 139 of stop cock 130 is positioned to the front, or upwards when stop cock 130 is positioned on the left handed side of introducer sheath 100. In the illustrated embodiment, rotatable knob 135 is positioned to the back, or downwards when stop cock 130 is positioned on the left handed side of introducer sheath 100, making manipulation of stop cock 130 difficult.

Where rotatable knob 135 is positioned to the back or downwards during a procedure, the practitioner must unnaturally twist his/her wrist to manipulate stop cock 130. The resulting unnatural positioning of the user's wrist and/or hand renders manipulation of stop cock 130 difficult and disadvantageous. In some circumstances, to avoid such physical contortions during the procedure, the user will twist the flexible tubing from which entry line 120 is formed. However, twisting of entry line 120 can result in kinking or restriction of the flow of fluids and other materials through entry line 120. Generally, such kinking or restriction is discouraged as it can potentially interfere with proper operation of the entry line.

As will be appreciated by those skilled in the art, a similar situation can arise where a left handed stop cock is positioned on the right hand side of an introducer sheath. Because it is difficult, if not impractical, to exchange a right handed introducer sheath for a left handed introducer sheath during a medical procedure, rotation of the introducer sheath during a procedure can be problematic, even where such rotation is need for other reasons. Additionally, the use of right handed and left handed stop cocks can be problematic for a manufacture or distributor who must provide an introducer sheath assembly with an interchangeable stop cock and/or both a right handed and left handed stop cock. Additionally, it can require additional attention for a hospital or other medical service provider to ascertain prior to a procedure whether the stop cock will be used in primarily a left handed or right handed orientation and then interchange right handed and left handed stop cocks where needed. This can not only require attention to detail, but require specialized training, and additional costs for the procedure. Additionally, it introduces the potential for contamination where such interchange is not properly performed.

### BRIEF SUMMARY OF THE INVENTION

The present invention relates to a stop cock for use with an introducer sheath that can be utilized in both left handed and right handed orientations. According to one embodiment of the present invention, the stop cock is utilized in combination with a rotator which allows the user to manipulate the rotatable knob of the introducer sheath in both left and right handed orientations. According to another embodiment of the present invention, the stop cock includes an integral rotator that allows manipulation of the positioning of the rotatable knob relative to the entry lines of the introducer sheath.

An introducer sheath includes a main body, a catheter or stem (also referred to herein as “sheath”), and one or more entry lines. At least a proximal portion of an entry line extends from the introducer sheath main body, and interfaces with a stop cock. The stop cock can include a delivery means disposed at one face of the stop cock, and can also interface with a second entry line. A rotator can interface between the first entry line and stop cock so that the stop cock can be rotated into an upward position regardless of which side of the patient's body the introducer sheath is mounted. In one embodiment, a second rotator can also interface between the stop cock and a distal portion of the entry line. Accordingly, an introducer sheath in accordance with an embodiment of the present invention can be easily configurable for both left and right handed users, and can be adapted to various spatial constraints with minimal effort.

The rotatable stop cock allows a manufacturer or distributor to only ship one stop cock for use with the introducer sheath. During preparation for the procedure, the stop cock does not need to be interchanged based on the predominant hand of the practitioner performing the procedure. Instead, the persons preparing for the procedure can turn their time and attention to other matters. In emergency or time sensitive procedures eliminating the need to interchange stop cocks can reduce the amount of time before a procedure can be started. Additionally, because the stop cock does not need to be interchanged, the sterile nature of the materials can be maintained. The manufacturer need provide only a single rotatable stop cock that can remain attached from the time of manufacture until the procedure is completed and the stop cock is discarded. During the procedure, in the event that the practitioner needs to rotate the introducer sheath, the stop cock can be quickly and efficiently rotated to maintain advantageous positioning of the rotatable knob of the introducer sheath throughout the procedure. This allows the user to quickly, efficiently, and correctly manipulate the knob without needing to twist or kink the tubing.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1A illustrates a perspective view of a prior art depiction of an introducer sheath in a first position;

FIG. 1B illustrates a perspective view of a prior art depiction of an introducer sheath in a second position;

FIG. 2A illustrates an introducer sheath utilized in combination with a rotatable stop cock according to one embodiment of the present invention.

FIG. 2B illustrates a rotator component of a rotatable stop cock according to one embodiment of the present invention.

FIG. 2C illustrates the introducer sheath of FIG. 2A utilized in combination with the rotatable stop cock of FIG. 2A in which the rotatable stop cock has been rotated subsequent to rotation of the introducer sheath.

FIG. 3A illustrates an introducer sheath utilized in combination with a rotatable stop cock assembly having first and second rotator components according to one embodiment of the present invention.

FIG. 3B illustrates an introducer sheath utilized in combination with a rotatable stop cock assembly having first and second rotator components utilized in connection with the entry lines according to one embodiment of the present invention.

FIG. 4 is a cross-sectional view illustrating a rotator component for use with a stop cock according to one embodiment of the present invention.

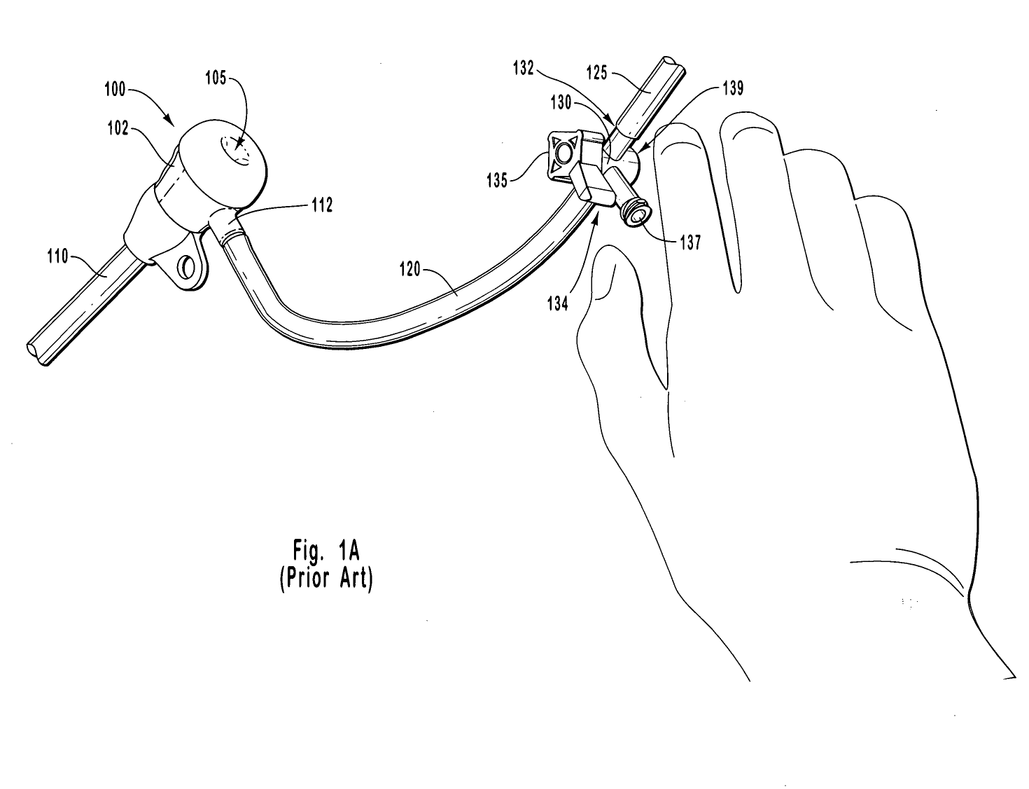

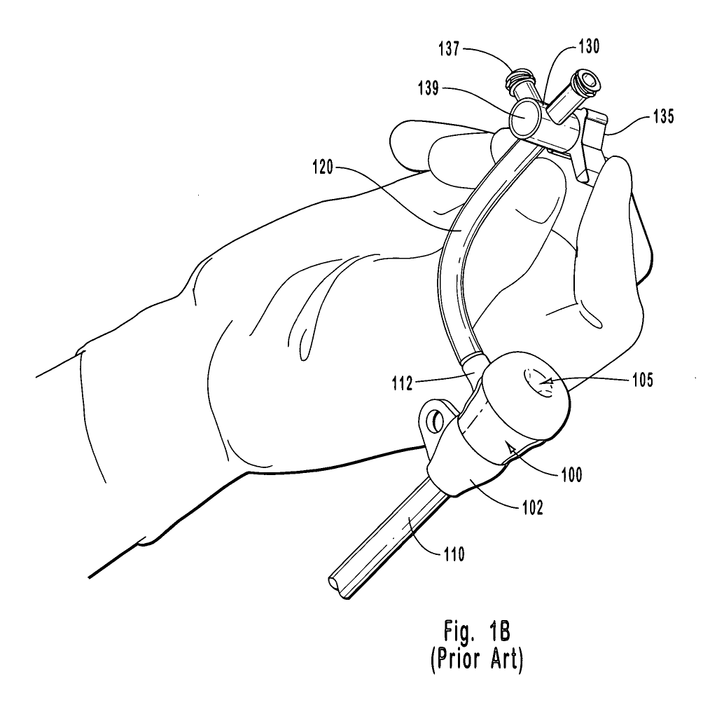

### DETAILED DESCRIPTION OF THE PREFERRED EMBODIMENTS

The present invention relates to a stop cock for use with an introducer sheath that can be utilized in both left handed and right handed orientations. According to one embodiment of the present invention, the stop cock is utilized in combination with a rotator which allows the user to manipulate the rotatable knob of the introducer sheath in both left and right handed orientations. According to another embodiment of the present invention, the stop cock includes an integral rotator that allows manipulation of the positioning of the rotatable knob relative to the entry lines of the introducer sheath. During the procedure, in the event that the practitioner needs to rotate the introducer sheath, the stop cock can be quickly and efficiently rotated to maintain advantageous positioning of the rotatable knob of the introducer sheath throughout the procedure. This allows the user to quickly, efficiently, and correctly manipulate the knob without needing to twist or kink the tubing.

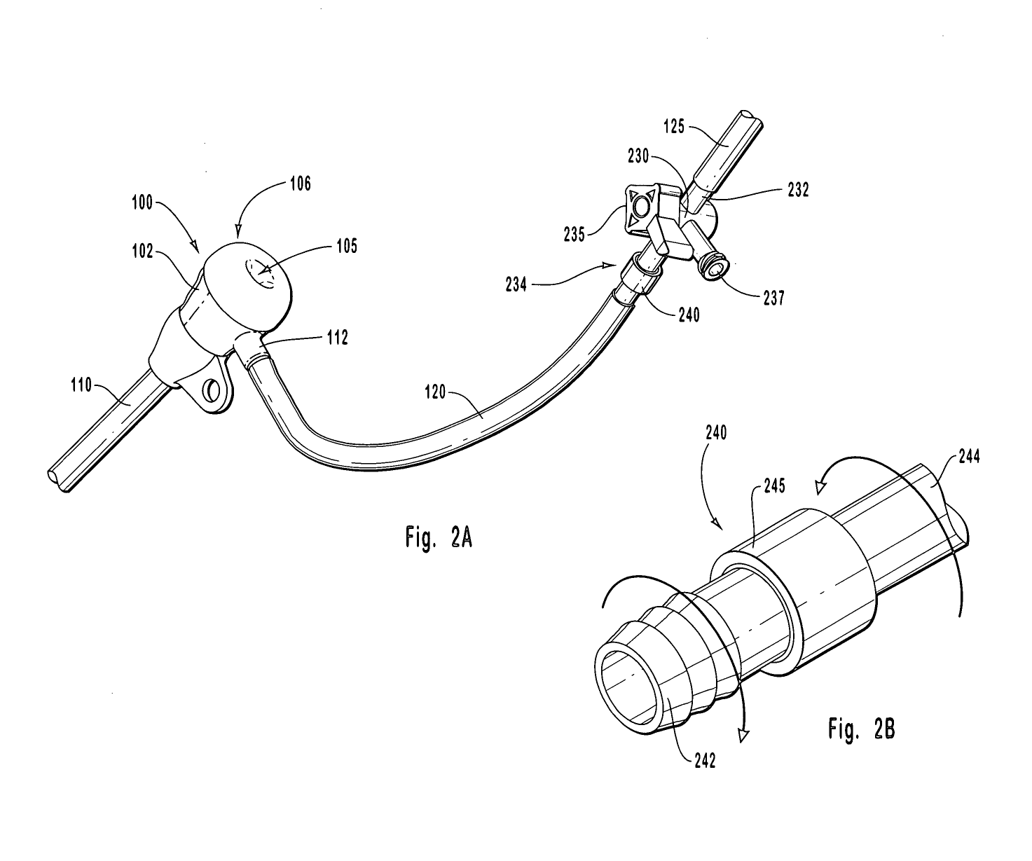

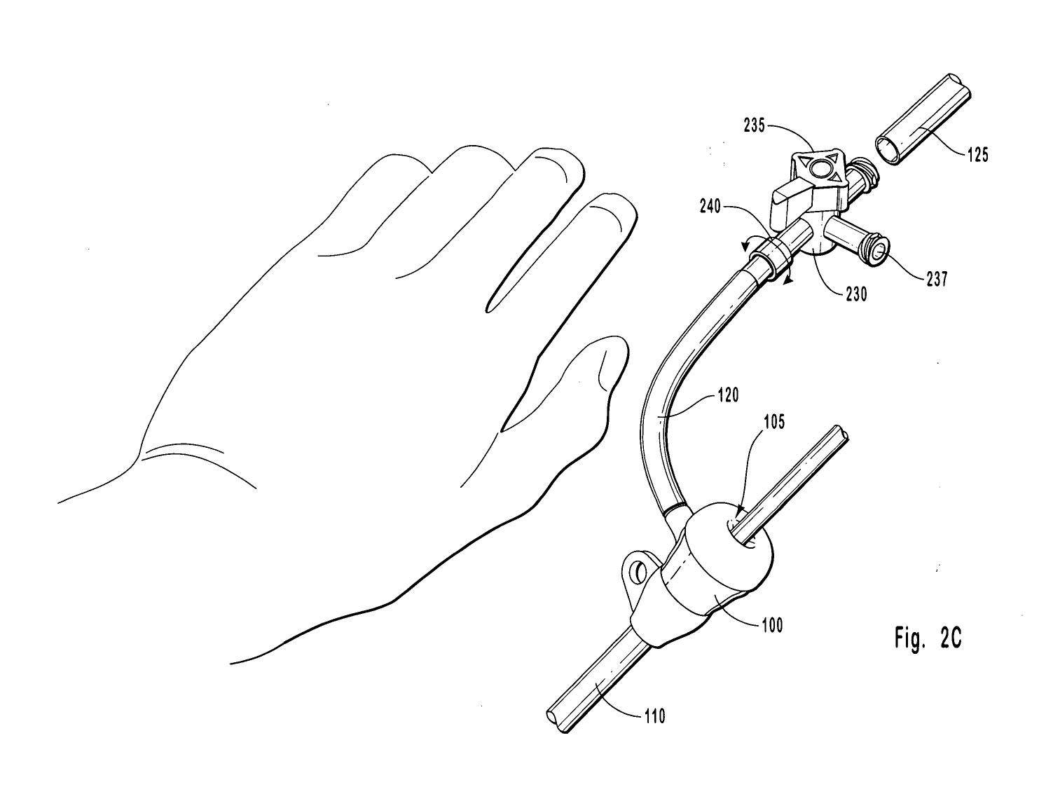

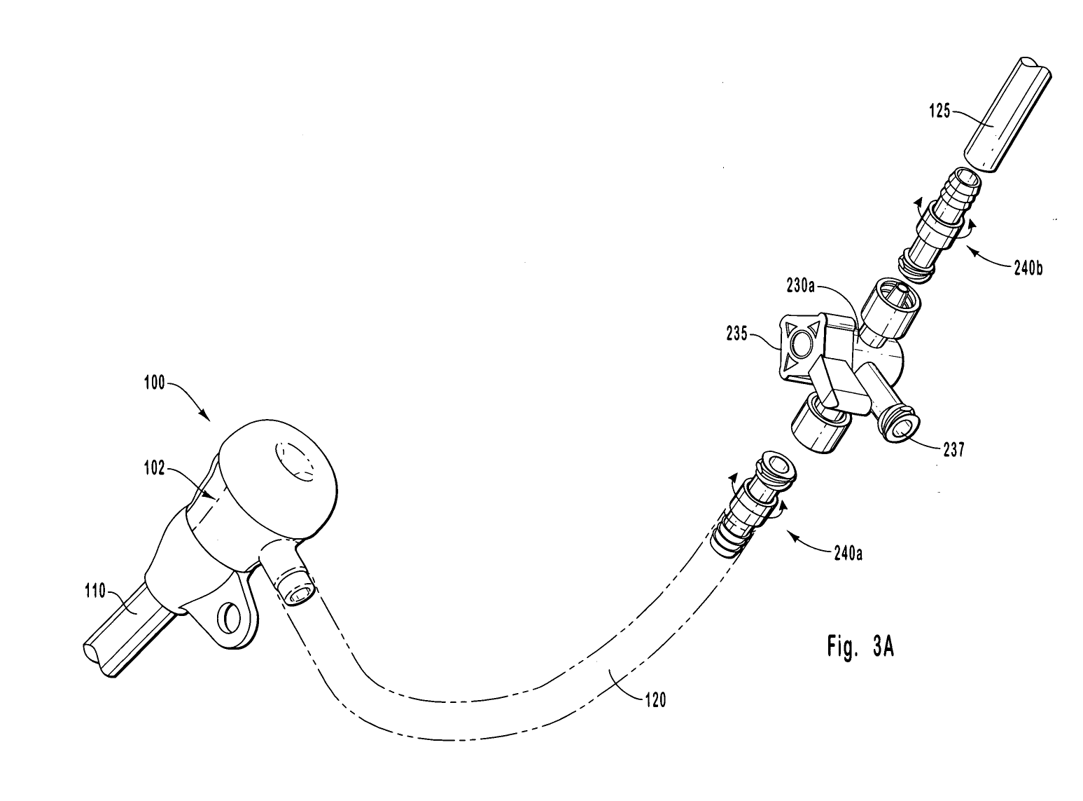

FIG. 2A illustrates an introducer sheath 100 for use in combination with a rotatable stop cock 230 according to one embodiment of the present invention. In the illustrated embodiment, introducer sheath 100 provides access to the vasculature or other internal portion of a patient's body. A portion of introducer sheath 100 is inserted into the patient's artery, vein, or other desired location. A practitioner can then insert articles, such as a guide wire or catheter, and materials, such as medicines or saline, through introducer sheath 100 and into the patient.

Introducer sheath 100 comprises a main body 102, an introducer bore 105, a hemostasis valve (not shown), a main lumen (not shown), a sheath 110, and a sideport 112. Introducer main body 102 provides a housing for securing the other components of introducer sheath 100. An introducer bore 105 is formed in a proximal end 106 of introducer sheath 100. A practitioner introduces guide wires, catheters, stents, balloons, and other articles and/or materials to be introduced into the patient through introducer bore 105. The hemostasis valve (not shown) is positioned internally at the proximal end 106 of introducer sheath 100 at a position adjacent to introducer bore 105 inside main body 102. The hemostasis valve provides a fluid seal between introducer bore 105 and the distal portions of introducer sheath 100. The hemostasis valve allows for the bloodless exchange of fluids and materials that are introduced into introducer sheath 100 through introducer bore 105.

The main lumen (not shown) provides a conduit through main body 102 of introducer sheath 100. The main lumen allows materials and/or fluids to pass through main body 102 and be introduced into sheath 110. Sheath 110 is configured to be inserted into an artery, vein, or other portion of a patient. Sheath 110 has an inner lumen of sufficient cross section to allow for the passage of catheters, stents, balloons, or other articles and materials utilized during a particular procedure to be performed. Side port 112 provides an additional entry point for introducing blood, saline, medicines, or other articles and materials into the patient through introducer sheath 100. Side port 112 feeds into the main lumen allowing passage of materials into the patient through sheath 110.

When a practitioner introduces a guidewire into the patient, the guidewire is first introduced through introducer bore 105. The guidewire is then threaded through the hemostasis valve and into the main lumen of the main body of the introducer sheath. The guidewire is threaded along the length of the main lumen and into sheath 110. The guidewire passes along the length the sheath and into the artery, vein, cavity, or other tissue of the patient. Introducer sheath 100 allows the practitioner to exchange multiple articles and materials into and out of the patient in a bloodless fashion during a single procedure without requiring multiple punctures of the patient.

In the illustrated embodiment, a single entry line 120 is attached to introducer sheath 100 at side port 112. Entry line 120 is positioned between stop cock 230 and introducer sheath 100. An entry line 125 is positioned proximally above stop cock 230. Generally, entry line 120 can provide a delivery conduit that can interface with both side port 112 of introducer sheath 100 and rotator 240 of outlet port 234. Entry line 125 interfaces with inlet port 232 of stop cock 230. Accordingly, entry line 125, stop cock 230, and entry line 120 can provide a continuous flow of fluid from a source of saline, fluids, or medicines to the introducer sheath.

In general, an “entry line” will be understood to mean a flexible tubing (e.g., flexible surgical tubing), or a series of connected flexible tubing members, that serve as a fluid delivery vehicle from a medical source to a patient's body through the introducer sheath. For example, an “entry line” can comprise a proximal portion 120 and distal portion 125 as part of a whole. In addition, a “medical source” (not shown) can be any container for housing of medicinal or nutritional fluids such as, for example, an infusate bag containing saline solution, nutritional supplements, bodily fluids, medicines, and so forth.

As will be appreciated by those skilled in the art, a variety of different types and configurations of side ports and entry lines can be utilized without departing from the scope and spirit of the present invention. For example, in one embodiment an introducer sheath that utilizes multiple entry lines that are connected to the introducer sheath main body are utilized. Having multiple connections to the introducer sheath housing 100 can be useful in the event, for example, that the practitioner utilizes multiple fluids or apparatuses through the same introducer sheath during a single procedure.

Entry line 120 positions stop cock 230 in fluid communication with introducer sheath 100. Stop cock 230 allows a user to control the flow of fluids and materials to introducer sheath 100. In the illustrated embodiment, stop cock 230 comprises an inlet port 232, an outlet port 234, a rotatable knob 235, a side port 237, and a rear surface 239. Rotatable knob 235 can connect within stop cock 230 to a valve that allows a user to control the flow of fluid into the introducer sheath, and ultimately into the patient's body. Thus, for example, if a user turns the knob 235 one fourth counter-clockwise, stop cock 230 can allow delivery of fluid. If the user turns knob 235 one quarter clockwise, the stop cock stops the flow of fluid. If the user turns knob 235 an additional one quarter clockwise, the stop cock allows venting from entry line 120 to the external environment.

In the illustrated embodiment, stop cock 230 comprises a rotatable stop cock. Stop cock 230 includes a rotator 240 that allows stop cock 230 to rotate about a perpendicular axis to allow facile and advantageous access to rotatable knob 235. In the illustrated embodiment, rotator 240 is integrally connected with outlet port 234 of stop cock 230. Rotator 240 allows stop cock 230 to be rotated relative to at least one of entry lines 120 and 125. Rotation of stop cock 230 allows a single stop cock to be utilized in both right handed and left handed configurations.

The rotatable stop cock allows a manufacturer or distributor to provide one stop cock for use with the introducer sheath. The manufacturer need provide only a single rotatable stop cock that can remain attached from the time of manufacture until the procedure is completed and the stop cock is discarded. During preparation for the procedure, the stop cock does not need to be interchanged based on the predominant hand of the practitioner performing the procedure. Instead, the persons preparing for the procedure can turn their time and attention to other matters. In emergency or time sensitive procedures, eliminating the need to interchange stop cocks can reduce the amount of time before a procedure can be started. Additionally, because the stop cock does not need to be interchanged, the sterile nature of the materials can be maintained. During the procedure, in the event that the practitioner needs to rotate the introducer sheath, the stop cock can be quickly and efficiently rotated to maintain advantageous positioning of the rotatable knob of the introducer sheath throughout the procedure. This allows the user to quickly, efficiently, and correctly manipulate the knob without needing to twist or kink the tubing.

As will be appreciated by those skilled in the art, a variety of types and configurations of rotatable stop cocks can be utilized without departing from the scope and spirit of the present invention. For example, in one embodiment, the stop cock can rotate relative to entry line 120 and 125 so that the side port can turn from right to left, and vice versa. Stop cock 230 is one example of a variable delivery means.

With reference now to FIG. 2B, a rotator 240 is illustrated separate from the other components of stop cock 230 of FIG. 2A. Rotator 240 allows a practitioner or other user to rotate stop cock 230 relative to introducer sheath 100. This provides additional functionality not realized without the use of rotator 240. For example, the practitioner can rotate introducer sheath 100 from a right handed to a left handed position, or vice versa, by simply twisting stop cock 230. Additionally, the practitioner can manipulate the positioning of stop cock 230 to more advantageously position the components of stop cock 230 during changing of infusate bags, different steps in a medical procedure, or for other desired purposes.

In the illustrated embodiment, rotator 240 includes a distal end 242, a proximal end 244, and a rotation mechanism 245. Rotation mechanism 245 allows distal end 242 to rotate relative to proximal end 244. A user can effectuate rotation of distal end 242 relative to proximal end 244 by grasping the stop cock and the entry line to which rotator 240 is attached and twisting the stop cock relative to the entry line. As will be appreciated by those skilled in the art, a number of approaches can be utilized to effectuate operation of the rotation mechanism to rotate the distal end of the rotator relative to the proximal end of the rotator. For example, twisting of the entry lines on opposing sides of the stop cock can be utilized to rotate the proximal end of the rotator relative to the distal end.

In the embodiment illustrated in FIG. 2A, rotator 240 is integrally coupled to the other components of stop cock 230. Rotator 240 comprises all or a portion of outlet port 234. Distal end 242 is configured to be coupled to entry line 120. Proximal end 244 is configured to be coupled to the body of stop cock 230. The configuration of rotation mechanism 245 allows entry line 120 and distal end 242 to rotate relative to proximal end 244 of stop cock 230. This allows the user to manipulate stop cock 230 without twisting or kinking entry line 120.

In the illustrated embodiment distal end 242 and proximal end 244 of stop cock 230 comprise different types of attachment mechanisms. As will be appreciated by those skilled in the art, a variety of different types and configurations of rotators can be utilized without departing from the scope and spirit of the present invention. For example, in one embodiment, the rotator can be a separate physical device that includes a physical interface that inserts into an entry line and a physical interface that inserts directly into the stop cock. Alternatively, the rotator can be a mechanical extension of the stop cock that can be inserted into two or more entry lines. The rotator can also be designed to extend from, or be positioned between, the side port of the introducer sheath and the stop cock. Furthermore, the introducer sheath, the entry line, the rotator, and the stop cock can be formed as a single unit that cannot be separated into component parts. In one embodiment, the rotatable stop cock is utilized with a rotatable suture ring to provide the ability to rotate not only the stop cock but also the suture ring. This allows the user to both manipulate the position the stop cock is facing while also adjust the positioning of the suture ring for attachment to the patient or once the suture ring is attached to the patient to manipulate the position of the suture ring relative to the side port or other components of the introducer sheath. The disclosure of U.S. patent application Ser. No. 11/021,513, filed on Dec. 23, 2004, entitled “Rotatable Suture Ring,” attorney docket number 10927.293 relating to the rotatable suture ring is incorporated herein by reference in its entirety.

In short, there are a variety of embodiments for providing a rotatable stop cock between the entry lines 120, 125. At least one junction along the entry line and the introducer sheath provides a rotator for allowing manipulation of the stop cock relative to the introducer sheath 100. In particular, rotator 240 can allow for less than full rotation (i.e. 10-180 degrees) or continuous rotation, although other intermediate degrees of rotation can also be provided.

As described above, an entry line 120 of surgical tubing can be positioned between stop cock 230 and introducer sheath 100. An entry line 125 of surgical tubing can be positioned proximally above stop cock 230. Generally, entry line 120 can interface with both side port 112 of introducer sheath 100 and rotator 240 of outlet port 234. The entry line 125 interfaces with inlet port 232 of stop cock 230. Accordingly, entry line 125, stop cock 230, and entry line 120 can provide a continuous flow of fluid from a medical source to the introducer sheath.

FIG. 2C illustrates introducer sheath 100 in which side port 112 has been moved to a left handed configuration in combination with stop cock 230. In the illustrated embodiment, rotator 240 of stop cock 230 has been rotated 180 degrees to position rotatable knob 235 of stop cock 230 in an upward position. A catheter is being introduced into introducer bore 105 of introducer sheath 100. In some circumstances a right handed practitioner may prefer to control movement of the catheter with the practitioner's right hand. This may be desired, for instance, in procedures where proper placement of the catheter tip is particularly important or difficult. As a result, a right handed practitioner may simply rotate introducer sheath 100 as shown to control stop cock 230 with his/her left hand.

Once introducer sheath 100 is rotated such that side port 112 is positioned on the left side of the practitioner, the user can rotate stop cock 230 utilizing rotator 240. Rotation of stop cock 230 allows the user to easily view, grasp, and manipulate rotatable knob 235. This minimizes uncomfortable and disadvantageous positioning of the user's hand and/or wrist during the procedure. Controlling disadvantageous positioning of the user's hand and/or wrist can be particularly important where the user is using the dominant hand for other aspects of the procedure.

As previously discussed, side port 112 can be positioned such that the stop cock 230 is disposed to the right of the introducer sheath. For purposes only of illustration, this position side port 237 such that it extends in a certain direction, such as to the right. This position of stop cock 230 can be a natural position for a right handed user to access the rotatable knob 235, since knob 235 is located on the face of the stop cock valve 230. The described rotation capability of the stop cock valve allows a high degree of configurability and positioning with the present introducer sheath. This allows the introducer sheath to be particularly suited to both left and right handed users with minimal interference to a patient.

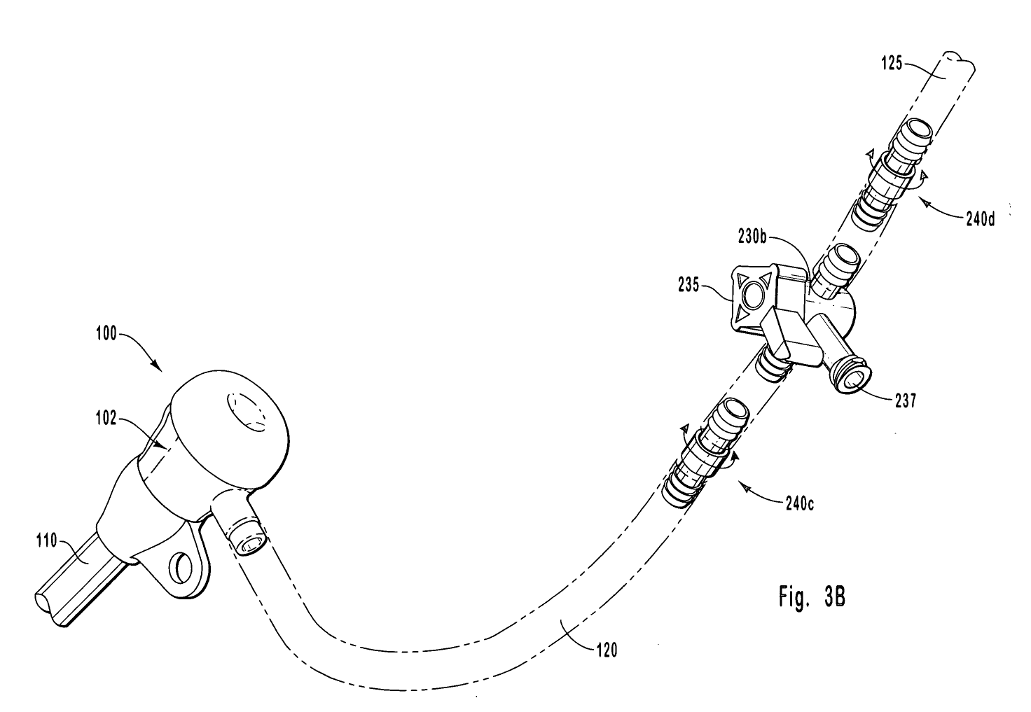

FIG. 3A is a perspective view of a stop cock 230a having a first rotator 240a and a second rotator 240b. In the illustrated embodiment, first rotator 240a is positioned at the distal end of stop cock 230a while second rotator 240b is positioned at the proximal end of stop cock 230a. First rotator 240a allows stop cock 230a to rotate relative to entry line 120. Second rotator 240b allows stop cock 230a to rotate relative to entry line 125. By allowing stop cock 230a to rotate relative to entry line 120 and entry line 125, first and second rotators 240a, b minimize twisting and kinking of the tubing delivering fluids and other materials to introducer sheath 100. In the illustrated embodiment, rotators 240a, b are integrally coupled to the other components of stop cock 230a.

The rotatable stop cock allows a manufacturer or distributor to provide a single stop cock for use with the introducer sheath. The manufacturer need provide only a single rotatable stop cock that can remain attached from the time of manufacture until the procedure is completed and the stop cock is discarded. During preparation for the procedure, the stop cock does not need to be interchanged based on the predominant hand of the practitioner performing the procedure. During the procedure, in the event that the practitioner needs to rotate the introducer sheath, the stop cock can be quickly and efficiently rotated to maintain advantageous positioning of the rotatable knob of the introducer sheath throughout the procedure. This allows the user to quickly, efficiently, and correctly manipulate the knob without needing to twist or kink the tubing.

FIG. 3B illustrates a stop cock 230b utilized in combination with first and second rotators 240c, d. In the illustrated embodiment rotators 240c, d are not integrally coupled to stop cock 230b. Rotators 240c, d are spaced apart from the body of stop cock 230b by small lengths of entry tubing. Rotators 240c, d allow stop cock 230b to be rotated relative to entry lines 120 and 125. The displacement between rotators 240c, d provides additional flexibility to the rotator stop cock assembly without substantially affecting the rotating ability of stop cock 230b.

As will be appreciated by those skilled in the art, a variety of different types and configurations of rotator stop cock assemblies can be utilized without departing from the scope and spirit of the present invention. For example, in one embodiment the rotators are integrally coupled to the stop cock utilizing the small lengths of flexible tubing. In another embodiment, the rotational ability of the stop cocks is unlimited. In yet another embodiment, the rotator stop cock assembly configures an introducer sheath and stop cock valve for left and right handed access. In another embodiment, the rotator stop cock assembly is utilized with an introducer sheath adapted to include multiple entry lines, and/or multiple rotators adjoined with multiple stop cock valves, as appropriate. The present invention therefore can provide a high degree of configurability without a need to purchase multiple right handed or left handed stop cocks.

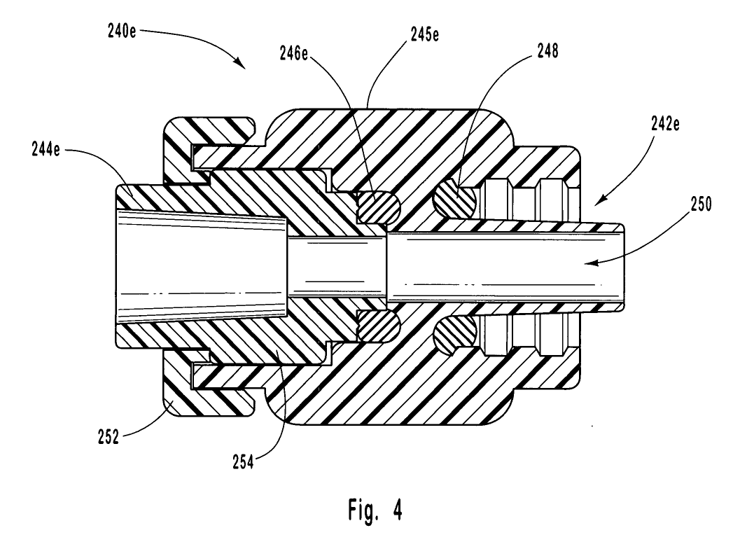

FIG. 4 is a cross-sectional side view of a rotator 240e illustrating a rotation mechanism 254e in greater detail. Rotator 240e is utilized with a stop cock to allow the stop cock to be optimally utilized in both right handed and left handed configurations as described with reference to FIGS. 2A-3B. In the illustrated embodiment, rotator 240e comprises a distal end 242e, a proximal end 244e, a rotation mechanism 245e, a rotator O-ring 246, a thread O-ring 248, a main lumen 250, and an end assembly member 252. Rotation mechanism 245e allows rotation of distal end 242e relative to proximal end 244e. In the illustrated embodiment, rotation mechanism 245e is provided by the juxtaposition of the body of distal end 242e and the body of proximal end 244e.

The body of distal end 242e is adapted to accommodate a portion of the body of proximal end 244e. The outer surface of the body of proximal end 244e includes a rotation flange 254. Rotation flange 254 contacts the inner surface of the body of distal end 242e such that proximal end 244e can rotate relative to distal end 242e. End assembly member 252 is secured-relative to the body of distal end 242e to maintain the position of rotation flange 254 relative to distal end 242e. This maintains the positioning of proximal end 244e relative to the body of distal end 242e.

Rotator O-ring 246 is positioned at the junction between the body of proximal end 244e and the body of distal end 242e. Rotator O-ring 246 provides a fluid tight seal to minimize the leakage of fluid from main lumen 250 to the exterior of rotator 240e. Additionally, rotator O-ring 246 provides a low-friction sliding surface to maintain smooth and efficient rotation of proximal end 244e relative to distal end 242e. Thread O-ring 248 is positioned at the base of the threads of distal end 242e. When a user threadably secures a stop cock, device, or implement to distal end 242e a surface of the stop cock, device, or implement abuts the thread O-ring 248. The contact between the surface of the stop cock, device, or implement provides a fluid tight seal between rotator 240e and the stop cock, device, or implement to which rotator 240e is secured.

As will be appreciated by those skilled in the art, a variety of types and configurations of rotators can be utilized in connection with the rotator stop cock assembly without departing from the scope and spirit of the present invention. For example, in one embodiment the rotator is adapted to be integrally secured to the stop cock. In another embodiment, the rotator utilizes a rotation mechanism that is a separate and distinct component from the body of the proximal and distal ends of the rotator. In another embodiment, the rotator utilizes a different combination of coupling components than those illustrated in FIG. 4.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within, their scope.

I claim:

1. A rotatable stop cock intended for use with an entry line and an outlet line, the rotatable stop cock comprising:
a main body having a valve assembly;
an inlet port positioned at one end of the main body and defining an entry lumen in communication with the valve assembly;
an outlet port positioned at the other end of the main body and defining an outlet lumen in communication with the valve assembly;
at least one rotator coupled to one or more of the inlet port and/or outlet port to allow the stop cock to rotate relative one or more of the entry and/or exit lines without twisting or kinking the entry and exit lines.

2. The rotator stop cock assembly of claim 1, wherein the one or more rotators comprise a portion of one or more of the inlet port and the outlet port.

3. The rotator stop cock assembly of claim 1, wherein the one or more rotators are integrally coupled to one or more of the inlet port and the outlet port.

4. The rotator stop cock assembly of claim 1, wherein the one or more rotators are indirectly linked to one or more of the inlet port and the outlet port.

5. The rotator stop cock assembly of claim 1, wherein the one or more rotators comprise a single rotator.

6. The rotator stop cock assembly of claim 1, wherein the one or more rotators comprise a first and second rotator.

7. The rotator stop cock assembly of claim 6, wherein the first and second rotators are positioned on opposing ends of the stop cock.

8. The rotator stop cock assembly of claim 1, wherein the stop cock utilized in combination with the one or more rotators comprises a rotatable stop cock.

9. The rotator stop cock assembly of claim 8, wherein the rotator stop cock assembly includes at least a second rotatable stop cock.

10. An introducer sheath comprising:
a main body defining an introducer bore formed in a proximal surface of the main body through which articles and materials can be inserted, the main body including a main lumen passing from the introducer bore to a distal end of the main body for facilitating the passage of the articles and materials through the main body, wherein the main body includes a right side and a left side;
a hemostasis valve positioned in the main lumen adjacent the introducer bore and providing bloodless exchange of articles and materials into the main lumen of the main body;
a tubular sheath positioned in fluid communication with the main lumen and extending from the distal end of the main body;
a side port coupled to the main body such that the side port can be positioned on one of the right side and the left side based on the rotational position of the main body, the side port providing a fluid passageway from the exterior of the main body to the main lumen such that fluid can be introduced to the main body from the side port;
an entry line tubing coupled in fluid communication to the side port to allow the introduction of fluid and materials from a fluid source to the side port;
a delivery valve in fluid communication with the entry line tubing and having a handle for operating a valve mechanism of the delivery, the handle having a first position for permitting the flow of fluids through the entry line tubing and a second position for stopping the flow of fluids through the entry line tubing; and
a rotator communicably coupled to the entry line and the delivery valve, such that the delivery valve is rotatable relative to the main body allowing the handle of the delivery valve to be accessed in both a right handed configuration and a left handed configuration when the side port and delivery tubing are positioned on either the right side of the main body or the left side of the main body.

11. The introducer sheath as recited in claim 10, wherein the delivery valve comprises a stop cock.

12. The introducer sheath as recited in claim 11, wherein the valve mechanism comprises an internal valve.

13. The introducer sheath as recited in claim 12, wherein the handle controls operation of the internal valve.

14. The introducer sheath as recited in claim 10, wherein the entry line comprises a first entry line and a second entry line.

15. The introducer sheath as recited in claim 14, wherein the delivery valve has one or more ports for receiving one or more fluid sources through one or more corresponding entry lines.

16. The introducer sheath as recited in claim 14, wherein the rotator is connected between the first entry line and the introducer sheath.

17. The introducer sheath as recited in claim 14, wherein the rotator is connected between the delivery line and the first entry line.

18. The introducer sheath as recited in claim 17, further comprising a second rotator that is connected between a second entry line and the stop cock, such that there are two rotators between a fluid source and the main body.

19. The introducer sheath as recited in claim 10, wherein the housing includes a plurality of side ports for receiving a plurality of entry lines.

20. The introducer sheath as recited in claim 10, wherein the delivery valve includes two or more stop cock ports, and wherein at least one of the two or more stop cock ports receives one or more of fluid from a fluid source, and the outside environment.

21. An rotatable stop cock comprising:
a main body;
an inlet port positioned at one end of the main body and linked to a delivery line;
an outlet port positioned at the other end of the main body and linked to a delivery line;
a rotation means for allowing rotation of the main body to rotate relative one or more delivery lines.

22. The introducer sheath as recited in claim 21, wherein the introducer sheath further comprises a second rotation means, wherein the one or more tubing components are connected to the rotation means and the second rotation means.

23. The rotatable stop cock of claim 21, wherein the rotation means comprises a rotation mechanism.

24. The rotatable stop cock of claim 21, wherein the rotation means comprises a rotator for use with the stop cock.

25. A rotatable stop cock comprising:
a main body;
an inlet port positioned at one end of the main body and linked to a delivery line;
an outlet port positioned at the other end of the main body and linked to a delivery line;
one or more rotators configured to allow the main body to rotate relative one or more delivery lines.
